(19) United States
(12) Patent Application Publication
(10) Pub. No.: US 2009/0177044 A1
(43) Pub. Date: Jul. 9, 2009

(54) DISPOSABLE SPECULUM

(76) Inventors: Yoram Cohen, Shoham (IL); Gadi Porat, Jerusalem (IL); Joel Stern, Herzliya (IL); Tamir Berman, Har Adar (IL)

Correspondence Address: PAUL D. BIANCO;Fleit Gibbons Gutman Bongini & Bianco PL, 21355 EAST DIXIE HIGHWAY, SUITE 115, MIAMI, FL, 33180, US

(21) Appl. No.: 12/298,145

(22) PCT Filed: Apr. 22, 2007
(86) PCT No.: PCT/IL07/00502

§ 371 Date: Jan. 27, 2009

Related U.S. Application Data
(60) Provisional application No. 60/794,112, filed on Apr. 24, 2006.

(51) Int. Cl. A61B 1/32

(52) U.S. Cl. 600/220

(57) ABSTRACT

A disposable plastic vaginal speculum constructed for heavy duty use and/or for long duration gynecological procedures including intrauterine device insertion and vaginal surgical procedures. The hinge region of the speculum, as well as other regions, are reinforced so that the speculum can bear forces of a magnitude normally bearable only by metal vaginal specula.

### FIELD OF THE INVENTION

The present invention relates to a sturdy disposable vaginal speculum.

### BACKGROUND OF THE INVENTION

Disposable, plastic vaginal specula are known. They are often formed of a moldable synthetic resin, such as polystyrene or polypropylene, and can be transparent or opaque, clear or colored.

Disposable vaginal specula are particularly useful for high-volume users, such as clinics which specialize in the detection of venereal disease or cervical cancer. Because of the large number of women being examined, it is advantageous to have a low-cost, disposable instrument obviating the need for cleaning and sterilization of the instrument after use. This is particularly true with the increase in occurrence of sexually transmitted diseases such as AIDS.

Generally, lubricants are required to facilitate insertion of the speculum. This can be problematic because lubricants often spoil pap smears and interfere with their cytological examination. In this regard, a plastic speculum is again preferred over a metal speculum because water or a simple silicone solution may be used as a lubricant with plastic specula reducing testing complications.

Because the practitioner or clinician should have both hands free during an examination, the vaginal speculum is preferably lockable at any of several open positions, and then easily released for removal. However, there are problems in constructing plastic specula which can reliably remain in an open, locked position. When pressure is exerted on the speculum's blades by the vaginal wall, the locking parts or blades of the speculum can distort. It is known that plastic specula do not possess the required mechanical strength for all medical procedures. Intrauterine devices (IUDs), for example, are generally not inserted using plastic specula because of their tendency to fracture at loads greater than about 2 to 3 kg. The fractured plastic generates sharp pieces with a concomitant risk of injury to the patient.

Another problem of prior-art disposable vaginal specula is that they often produce noisy clicks when being opened within the patient. This noise is caused by the ratchet action of the speculum's locking mechanism. Although the ratchet action itself is not harmful, the clicking noise is often disturbing to the patient. It sometimes causes an involuntary response on the part of the patient, often leading to sudden increases in pressure on the speculum causing it to break.

Typical plastic vaginal specula are disclosed, for example, in U.S. Pat. Nos. 3,568,665; 3,752,149; 3,246,646; 3,332,414; 3,650,266; 3,985,125, and 3,890,961.

In view of the above remarks it would be advantageous to develop a sturdy plastic vaginal speculum which would not fracture or collapse under loads normally encountered during examinations of long duration or during extended gynecological surgical procedures.

### SUMMARY OF THE PRESENT INVENTION

It is an object of the present invention to provide a sturdy disposable vaginal speculum that allows for the performance of long-duration medical procedures.

It is also an object of the present invention to provide a sturdy, safe, disposable vaginal speculum that allows introduction of an IUD into a patient and the performance of surgical procedures, in addition to performing conventional regular periodic gynecological examinations.

It is a further object of the present invention to provide a safe, substantially non-breakable, disposable speculum that can withstand loads in excess of about 2 to 3 kg.

There is thus provided in accordance with one aspect of the present invention a vaginal speculum including a molded plastic dorsal member having a rounded dorsal blade member. The blade member is formed integrally with a dorsal handle member. The dorsal member further includes a first hinge location formed on each of its lateral faces. The speculum also includes a molded plastic ventral member having a rounded ventral blade member formed integrally with a ventral handle member. The ventral member further includes a second hinge location formed on each of its lateral faces. The second hinge locations are for interconnecting with the first hinge locations thereby forming a hinge for facilitating relative angular translation between the dorsal member and the ventral member. The angular translation is about an axis extending transversely through the hinge and adapted to support shear force components normal to the axis. The speculum further includes a locking mechanism in mechanical communication with the dorsal and ventral handle members. The locking mechanism locks the dorsal blade member and the ventral blade member in a user-selected position with respect to each other. The hinge is reinforced so as to facilitate resistance to shear force components of a magnitude more normally associated with vaginal specula formed of metal.

In a further embodiment of the vaginal speculum, the locking mechanism further includes an at least partially threaded screw that is joined to and extends from the dorsal handle member through the ventral handle member. The mechanism has a nut positioned on the screw for holding the ventral and dorsal blade members in a predetermined user-selected position with respect to each other. The screw may have threads which form a profile chosen from a group of profiles consisting of a rectangular profile and a trapezoidal profile. The screw and nut may have one or more threaded regions for facilitating rapid locking and unlocking of the speculum. The screw may also include a head to prevent accidentally dislodging the nut.

In an additional embodiment of the speculum of the present invention where the locking mechanism is a screw and nut, the screw is positioned near the points of maximum bending of the blade members.

In yet another embodiment of the speculum of the present invention the plastic is a high stress resistant plastic suitable for supporting stresses in excess of at least a force of 6 kgs without bending, similar to metal specula. In some cases, the high stress resistant plastic is polycarbonate. The high stress resistant plastic may be a transparent plastic.

In still another embodiment of the speculum of the present invention, each of the first hinge locations includes a hinge pin and each of the second hinge locations includes a hinge hole. In some cases, the hinge pins are mushroom-head pins.

In a further embodiment of the vaginal speculum constructed according to the present invention, both of the lateral faces of the dorsal member proximal to the ventral member include a thickening of the plastic in the neighborhood around the hinge holes. In this embodiment, each of these lateral faces also having a hinge pin lead slot leading to the hinge holes from an edge of the dorsal member. The hinge pin lead slot is typically formed by side core injection molding.

In embodiments of the speculum of the present invention, the ventral member of the vaginal speculum may include one or more of the following structural features:
thickening of the plastic at a distal curved surface substantially at the point where the ventral blade member and the ventral handle member merge; and
thickening of the plastic at one or more proximal surfaces of the ventral member substantially at the point where the ventral handle member and the ventral blade member merge, the thickened portion located below the level of the hinge pins.
When these structural features are effected on the ventral member, the dorsal member may include one or more of the following structural features:
thickening of the plastic at a distal curved surface substantially at the point where the dorsal blade member and the dorsal handle member merge, the thickened portion located above the level of the hinge holes;
thickening of the plastic at one or more curved surfaces positioned below the level of the hinge holes on the distal surface of the dorsal member; and
struts positioned along the distal curved surface of the dorsal member substantially in the region where the dorsal blade member and the dorsal handle member merge.

In a further embodiment of the present invention, the ventral handle member further includes an extension so that when the ventral and dorsal handle members are brought together separating the ventral and dorsal blade members, the extension is supported by the dorsal handle member and the extension in turn supports the speculum by spreading the forces over the entire device. In this embodiment, the dorsal member further includes a support ridge above the hinge holes in the dorsal member, so that when the speculum is under extreme load the speculum does not fracture in the region of the holes and thereby does not endanger the patient.

In yet another embodiment of the vaginal speculum, each of the second hinge locations includes a hinge pin and each of the first hinge locations includes a hinge hole.

### DEFINITIONS

Ventral member—In the specification below this member of the speculum may also be described as the fixed member or the lower member.

Dorsal member—In the specification below, this member of the speculum may also be described as the movable member or the upper member.

Proximal—the direction closest to the user of the speculum.

Distal—the direction furthest from the user of the speculum

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in greater detail than is necessary for a fundamental understanding of the invention. The description taken with the drawings make apparent to those skilled in the art how the several forms of the invention may be embodied in practice.

In the drawings:

FIG. 1 is a side view of a disposable speculum constructed according to a first embodiment of the present invention, the speculum being in its closed configuration;

FIG. 2 is a front view of the blade profile of the disposable speculum of FIG. 1, the speculum being in its closed configuration

FIG. 3 is a side view of the disposable speculum of FIG. 1, the speculum being in its open configuration;

FIG. 4 is a schematic side view of the ventral member of the speculum appearing in FIGS. 1 and 3;

FIG. 5 is a schematic proximal view of the ventral member of the speculum appearing in FIGS. 1 and 3;

FIG. 6 is a schematic truncated side view of the proximal portion of the dorsal member of the speculum appearing in FIGS. 1 and 3;

FIG. 7 is a schematic proximal view of the dorsal member of the speculum appearing in FIGS. 1 and 3;

FIG. 8 is a top view of the dorsal blade of the speculum appearing in FIGS. 1 and 3;

FIG. 9 is a schematic side view of the dorsal member of the speculum appearing in FIGS. 1 and 3;

FIG. 10 is a prior art cross sectional view of the portion of the dorsal member which serves to connect with the ventral member;

FIG. 11A is a cross sectional view of the portion in the dorsal member which serves to connect with the ventral member in the speculum shown in FIGS. 1 and 3;

FIG. 11B is an isometric view of the portion in the dorsal member which serves to connect with the ventral member in the speculum shown in FIGS. 1 and 3;

FIG. 12A is a schematic side view of the ventral member of the speculum appearing in FIGS. 1 and 3;

FIG. 12B is a schematic proximal view of the ventral member of the speculum appearing in FIGS. 1 and 3;

FIG. 12C is a schematic side view of a mushroom-head hinge pin used in the speculum shown in FIGS. 12A and 12B;

FIG. 12D is a schematic top view of a mushroom-head hinge pin used in the speculum shown in FIGS. 12A and 12B;

FIG. 13 is a schematic side view of a disposable speculum constructed according to a second embodiment of the present invention;

FIG. 14 is a schematic side view of a dorsal member constructed according to a second embodiment of the present invention;

FIG. 15A shows a disposable speculum constructed according to a third embodiment of the present invention;

FIGS. 15B and 15C show a side view of the ventral member and a proximal view of the dorsal member, respectively, of the speculum shown in FIG. 15A;

FIG. 16A shows a threaded bolt or screw which may be used in the locking mechanism in embodiments of the present invention; and

FIGS. 16B-16E show different profiles of the threads which may be used with the bolts or screws of the locking mechanism of the present invention, some profiles being acceptable for use while others are not.

Similar elements in the Figures are numbered with similar reference numerals.

### DESCRIPTION OF PREFERRED EMBODIMENTS

Currently available disposable plastic vaginal specula are structurally too weak to be employed for anything other than routine short-duration examinations, including examinations where vaginal or cervical smears are obtained. In long-duration procedures, such as the introduction of an intrauterine device (IUD) and gynecological surgical procedures, the patient may reflexively or otherwise generate sudden large mechanical loads on the plastic device resulting in its fracturing. Additionally, if the plastic vaginal speculum must be held open for relatively long durations, blade bending and even collapse may occur. Changes are therefore required in the construction of plastic specula.

The present invention teaches the use of sturdier plastics with which to form plastic specula. Additionally, design enhancements intended to buttress potential points of failure of plastic specula have been introduced. Currently available plastic specula may support loads of up to about two to three kilograms. The present invention teaches a speculum that can support loads in excess of about six kilograms over sustained periods, similar to metal specula.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

It is to be appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination.

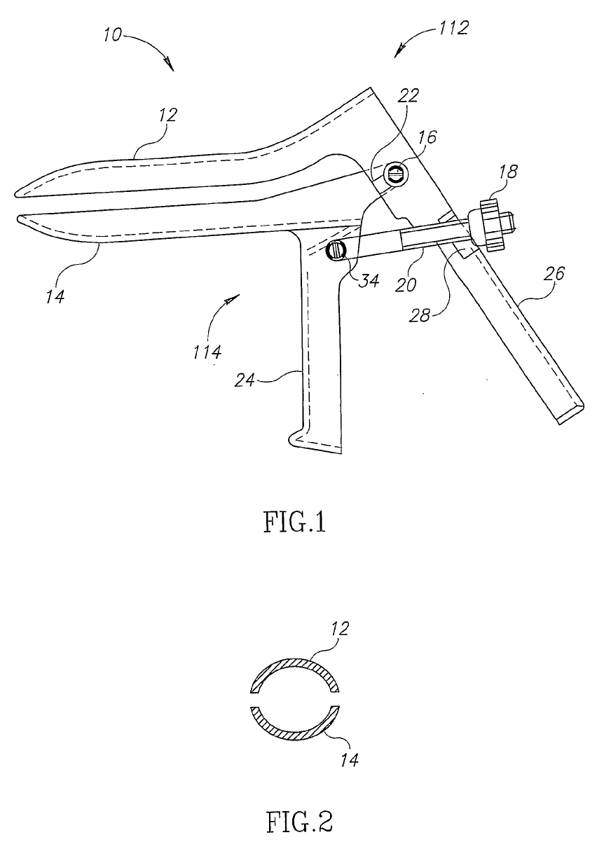

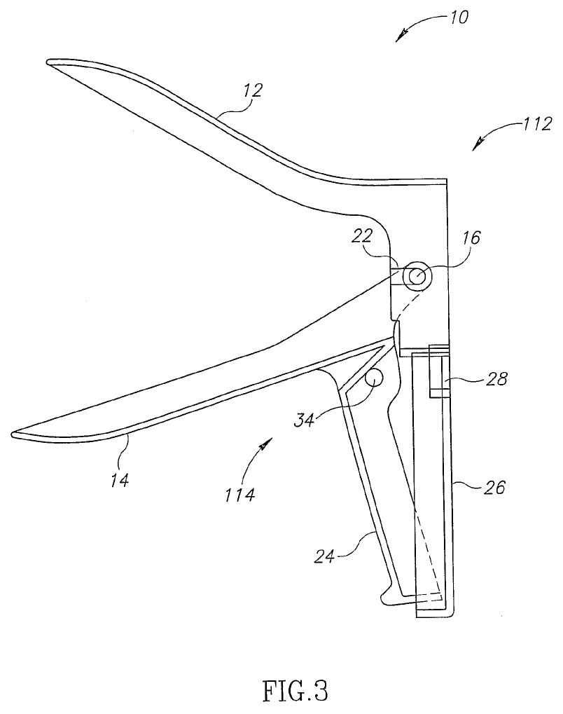

Reference is now made to FIGS. 1 and 3 which show side views of a disposable speculum 10 constructed according to a first embodiment of the present invention. FIG. 1 shows speculum 10 in its closed configuration and FIG. 3 shows speculum 10 in its open configuration. Speculum 10 is formed of injection molded plastic, in the present invention a high strength plastic, such as polycarbonate. Other resins possessing similar mechanical strength my also be used to form the specula of the present invention. In all events, the plastic must be stronger and more durable than the currently employed polystyrene or polypropylene. Typically, the plastic is a clear transparent plastic but colored or non transparent high strength plastics may also be used.

Speculum 10 is formed of a dorsal member 112 and a ventral member 114. The Dorsal member 112 contains a dorsal blade member 12, a dorsal handle member 26 integrally formed with blade member 12, a pair of hinge holes 30 (best seen below in FIG. 7 to be discussed below), one on each lateral side of dorsal member 112, and a dorsal member bolt hole 28 formed as an integral extension from handle member 26. In other embodiments, bolt hole 28 may be formed directly in handle member 26.

Ventral member 114 includes a ventral blade member 14, a ventral handle member 24 integrally formed with blade member 14, and integrally formed hinge pins 16, one on each lateral side of ventral member 114. Hinge pins 16 can also be formed as studs, trunnions and the like; they can be best seen in FIG. 5 to be discussed below. Hinge pins 16 are insertable into hinge holes 30 of dorsal member 112 thereby engaging members 112 and 114 for use. Hinge pins 16 and hinge holes 30 form a hinge around which dorsal blade member 12 pivots when speculum 10 is brought from its closed to its open configuration or vice-versa. In the closed configuration dorsal blade member 12 lies facing and immediately adjacent to ventral blade member 14.

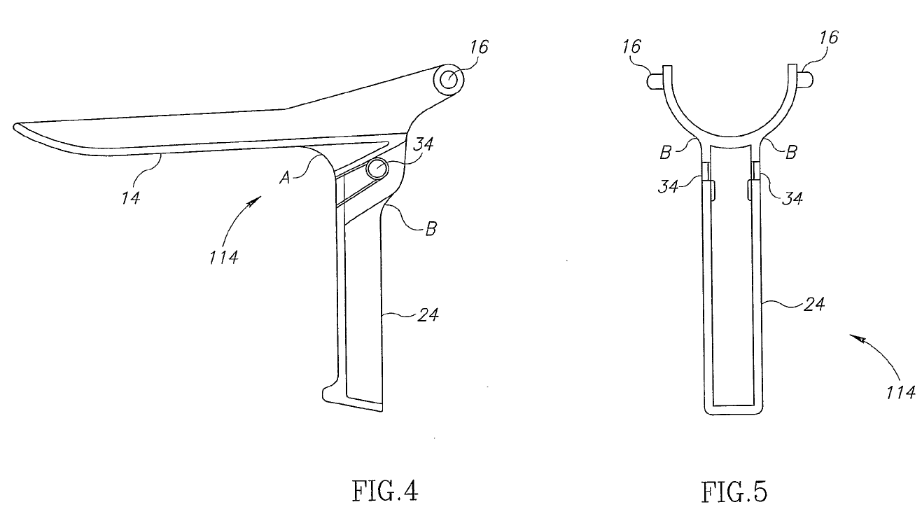

An at least partially threaded bolt or screw 20 extends into and through dorsal member bolt hole 28 and then into at least one bolt hole 34 (best seen in FIG. 4) in ventral handle member 24. In the embodiment of FIG. 4, bolt or screw 20 makes an approximately right angle turn after extending from dorsal member bolt hole 28, entering ventral member bolt holes 34 on ventral handle member 24. Without intending to be limiting the bolt or screw is typically made of a plastic, such as nylon, polyethylene, polypropylene, acetal (polyoxymethylene) and polycarbonate. The bolt or screw may have a splayed end which after passing through ventral member bolt holes 34 expands to engage and hold the bolt or screw to ventral handle member 24. Other methods of engagement known to those skilled in the art may also be used. In other embodiments, bolt or screw 20 may pass directly from dorsal member bolt hole 28 to a single ventral member bolt hole 34 without requiring a right angle turn.

Plastic nut 18 sits on the side of dorsal member bolt hole 28 distal from ventral handle member 24. After speculum 10 is brought from its closed configuration to its partially or completely open configuration, nut 18 is used to lock the handles in the position desired by the physician while he carries out the required gynecological procedures.

Nut 18 and threaded bolt or screw 20 together form a locking mechanism and may be described herein as such. It should be evident to one skilled in the art that a nut and bolt mechanism is not the only locking mechanism that can be used and therefore its use here should not be deemed to be limiting.

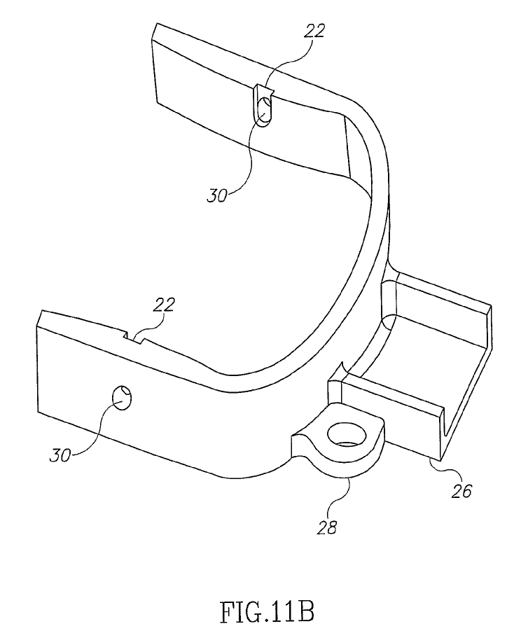

FIGS. 1 and 3 show a hinge pin lead slot 22 integrally formed on the inside lateral face of dorsal member 112. Slot 22 typically runs from hinge hole 30 to a point on the distal edge of dorsal member 112. Slot 22 will be discussed further in conjunction with FIG. 11 below.

In FIG. 2, to which reference is now made, a front profile of blade members 12 and 14 of speculum 10 discussed in conjunction with FIGS. 1 and 3 is shown. The blade members are in their closed configuration.

Reference is now made to FIGS. 4 and 5 which show side and proximal views, respectively, of ventral member 114 of speculum 10 discussed above in conjunction with FIGS. 1 and 3. All the elements present there have been discussed in conjunction with FIGS. 1 and 3 and therefore will not be discussed again.

Those regions shown in heavy black in FIGS. 4 and 5 and in other Figures discussed below indicate portions of the speculum reinforced by thickening the plastic walls.

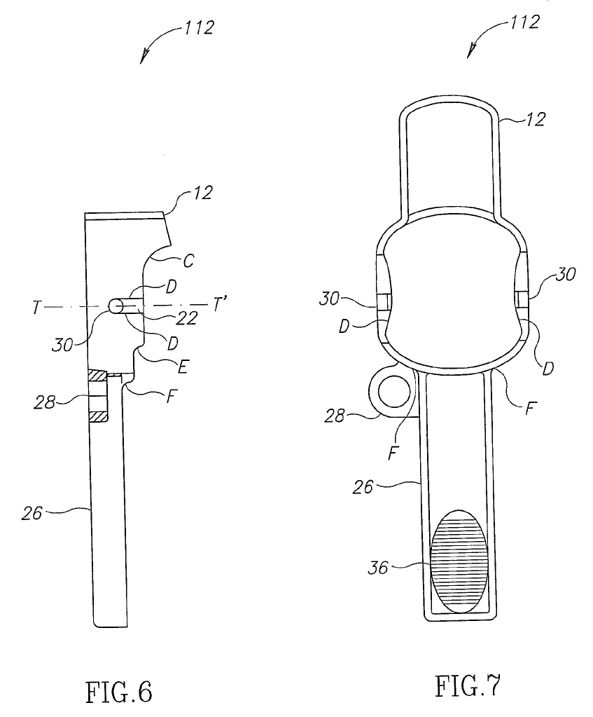

Reference is now made to FIGS. 6 and 7 which show truncated side and proximal side views respectively of dorsal member 112 of speculum 10 discussed above in conjunction with FIGS. 1 and 3. Most of the elements present in FIGS. 6 and 7 have been discussed in conjunction with FIGS. 1 and 3 and therefore will not be discussed again. In FIGS. 6 and 7, hinge holes 30 are clearly seen as is hinge pin lead slot 22, the latter being situated on the inside lateral face of dorsal member 112. The purpose of slot 22 is to allow ease of assembly and insertion of hinge pins 16 (FIG. 5) into hinge holes 30. In FIG. 7 finger rest 36 is also shown.

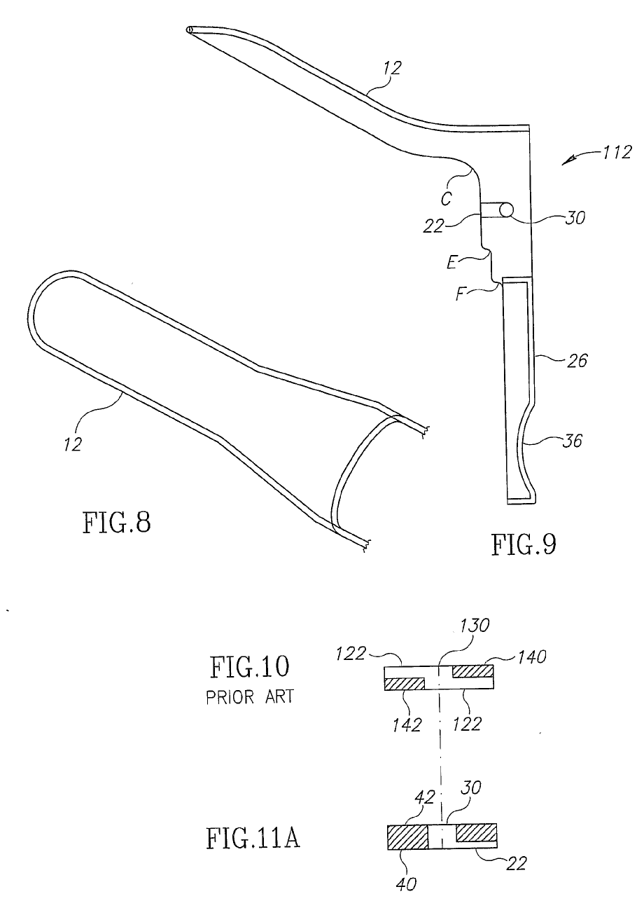

FIG. 8, to which reference is now made, shows a top view of dorsal blade member 12 of dorsal member 112 of FIGS. 1 and 3.

Reference is now made to FIG. 9 which shows a side view of dorsal member 112 which includes dorsal blade member 12, integrally formed with and joined to dorsal handle member 26, and hinge hole 30 of speculum 10 discussed above in conjunction with FIGS. 1 and 3. Since these elements have been discussed in conjunction with FIGS. 1 and 3 they will not be discussed again. In FIG. 9, finger rest 36 is also shown.

Turning now to FIGS. 10, 11A and 11B, hinge pin lead slots 122 in prior art specula and hinge pin lead slots 22 in the speculum of the present invention are shown. The views shown there are along line TT′ shown in FIG. 6. The prior art lead slots 122 (FIG. 10) are produced via injection molding using the steel joint method while the lead slot 22 (FIGS. 11A and 11B) of the present invention is produced by injection molding using the side core method. The purpose of slots 22 in the present invention is to permit ease of insertion of hinge pins 16 into hinge holes 30. Because there is more plastic in the region of hinge hole 30 using lead slot 22 of the present invention shown in FIGS. 11A and 11B, the probability of fracture through, or near, hinge hole 30 of the present invention, is reduced.

In FIG. 10 formed according to prior art methods, hinge hole 130 is at the hub of two hinge pin lead slots 122, one on the outside lateral face 142 of dorsal member 112 and one on the inside lateral face 140 of dorsal member 112. In FIGS. 11A and 11B, where lead slot 22 is formed according to the side core method, hinge hole 30 is at the hub of one hinge pin lead slot 22, this slot being positioned on the inside lateral face 40 of dorsal member 112. In the present invention, there is no slot present on the outside lateral face 42 of dorsal member 112.

The isometric view in FIG. 11B is presented for better understanding the position and shape of the single hinge pin lead slot 22 discussed in conjunction with FIG. 11A

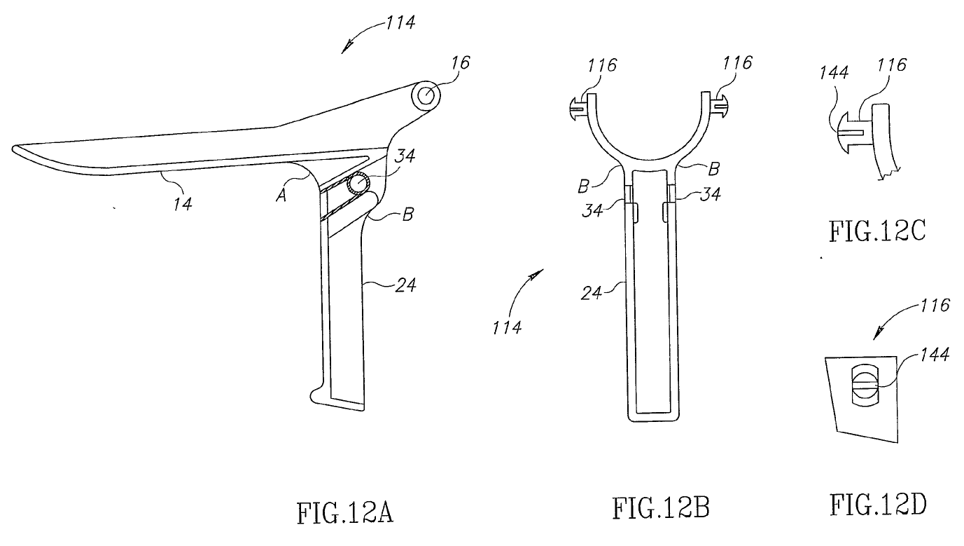

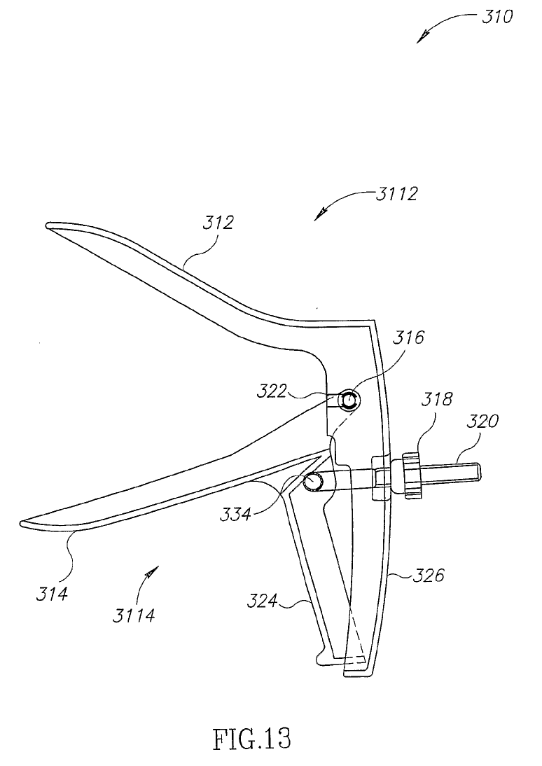

Reference is now made to FIGS. 12A-12D where a speculum constructed according to an embodiment of the present invention is shown. This embodiment is the same as that shown in FIGS. 1-9 but the hinge pins are mushroom-shaped hinge pins 116. FIG. 12A shows a schematic side view of ventral member 114 of the speculum and FIG. 12B shows a schematic proximal view of ventral member 114 of the speculum. FIG. 12C and FIG. 12D show side and top views, respectively, of the mushroom-shaped hinge pins 116 used in ventral member 114 of FIGS. 12A and 12B. For ease of insertion, the top of pin 116 includes a groove 144 which can accommodate a tool for manipulating and bringing pin 116 through the hinge holes (not shown) of a speculum's dorsal member (also not shown).

In another embodiment of the present invention shown in FIG. 13, to which reference is now made, the at least partially threaded bolt or screw 320 of speculum 310 is positioned higher up handle members 326 and 324, that is closer to the interface region between handle members 324 and 326 and blade members 312 and 314. By positioning screw or bolt 320 higher up handle members 326 and 324, bending of blade member 312 and 314 is reduced as is speculum failure. Except for the positioning of bolt or screw 320, speculum 310 shown in FIG. 13 is as shown in the embodiment presented previously in FIGS. 1-9. Accordingly, the numbering in FIG. 13 is identical with that presented in FIGS. 1-9 but with the addition of a prefix digit “3”.

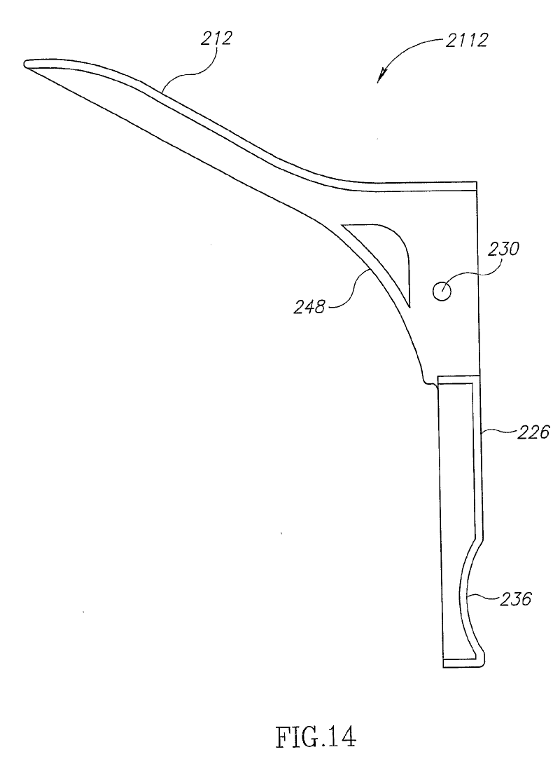

FIG. 14, to which reference is now made, shows a dorsal member 2112 constructed slightly differently from the dorsal members 112 shown previously. Member 2112 includes a pair of integrally formed struts 248 to buttress the distal region of curvature (that is, the distal face of the region where dorsal blade member 212 and dorsal handle member 226 merge) of dorsal member 2112. The remainder of member 2112 is similar to member 112 shown in FIGS. 1, 3 and 9, for example, and will not be discussed further. Parts of member 2112 similar to those in dorsal member 112 have been numbered similarly but with the addition of a first digit of “2”.

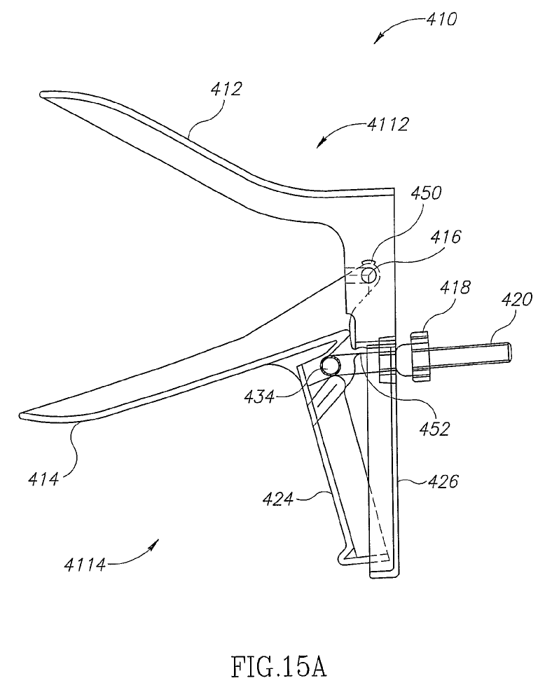

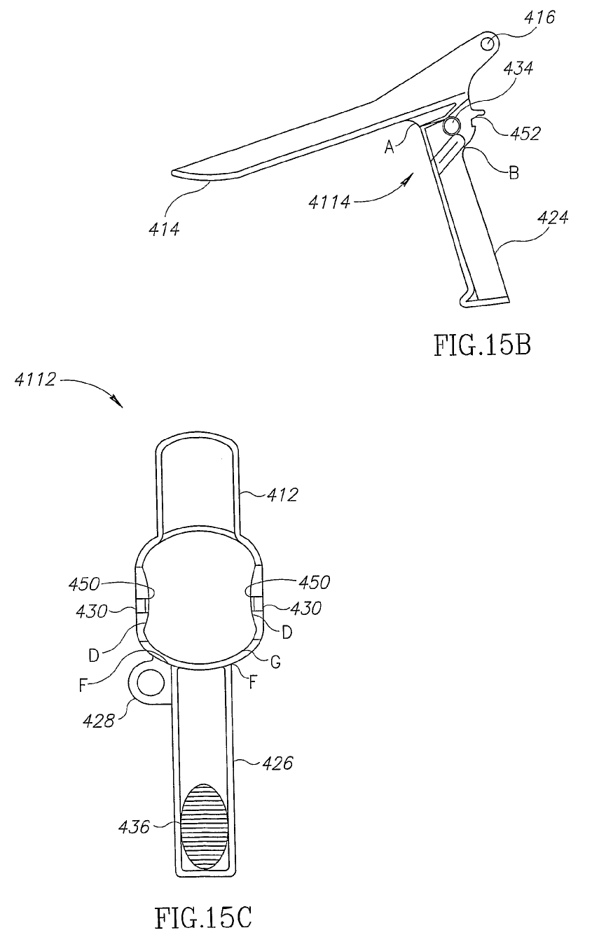

In FIGS. 15A to FIG. 15C a third embodiment of the present invention is shown wherein FIG. 15A shows a speculum 410 in its open configuration, FIG. 15B shows the ventral member 4114 of speculum 410, and FIG. 15C shows a proximal view of dorsal member 4112 of speculum 410.

This third embodiment is very similar to the previous embodiments and similar elements, members and parts have been given similar numbers as in FIGS. 1-9 but with a prefatory digit of “4”. Since they are similar a discussion of their operation will not be repeated. FIGS. 15A and 15B show an additional element, extension 452 extending in the proximal direction from the proximal edge of ventral handle member 424. Additionally, as seen in FIGS. 15A and 15C there is a ridge structure 450 above hinge hole 430. When speculum 410 is locked in an open or partially open position, extension 452 rests against and is supported by dorsal member handle 426. Extension 452 thereby can assist in supporting the speculum by spreading the forces over the entire device when the device is under load. Ridge structure 450 provides additional support for hinge pin 416 when speculum 410 is in its open loaded configuration.

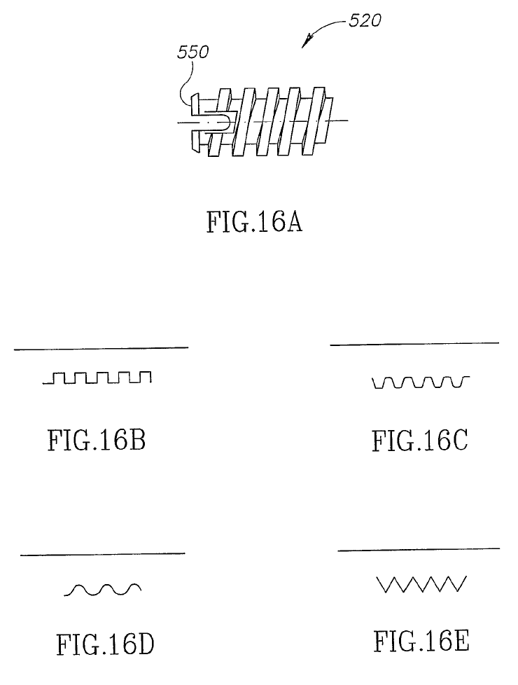

Reference is now made to FIGS. 16A-16E which show the threaded bolt or screw 520 which may be used in embodiments of the present invention, as well as various thread profiles. It has been found that trapezoidal (FIG. 16A and FIG. 16C) or rectangular (FIG. 16B) thread profiles impart greater strength to the bolt or screw compared to triangular (FIG. 16E) or sinusoidal (FIG. 16D) thread profiles. In embodiments of the present invention, the threaded bolt or screw locking mechanism uses rectangular or trapezoidal thread profiles. Additionally, the bolt or screw contains a head 550 (FIG. 16A) at its proximal end so that the nut positioned on the bolt or screw does not unintentionally drop off.

While what has been described herein has described a locking mechanism employing a threaded bolt or screw extending from the dorsal handle member to the ventral handle member, other locking mechanisms readily known to those skilled in the art can also be used. Additionally, we have described a bolt or screw mechanism that reaches from the dorsal handle member to the ventral handle member entering the latter from the side after making a right angle turn. It should readily be understood by one skilled in the art that a direct connection can be effected where the screw or bolt passes through a single integrally formed bolt hole on each of the handles without making a right angle turn.

It should be evident to one skilled in the art that while we have described the speculum of the present invention as having hinge pins on the ventral member and hinge holes on the dorsal member, the invention can be effected with a construction where the hinge pins are located on the dorsal member and the hinge holes on the ventral member.

Heavy black markings appear in FIGS. 4 through 7 and 9, to which reference is again made. These markings show areas of the dorsal 112 and ventral 114 members of speculum 10 that are strengthened by thickening the walls of the respective members at the indicated locations. These areas include: the area around distal curvature A of ventral member 114 and the proximal portion B of ventral member 114, approximately where ventral blade member 14 merges with ventral handle member 24. Similarly, the areas around distal curvature C of dorsal member 112 and the distal portions E and F of dorsal member 112 approximately where dorsal blade member 12 merges with dorsal handle member 26 are strengthened by thickening the plastic in those regions. As mentioned previously, thickened plastic is also present on the inside lateral face D of dorsal member 112 near hinge pin lead slot 22. Lead slot 22 allows for ease of assembly by the manufacturer.

By using combinations of two or more of the following structural enhancements a disposable speculum with a strength at least three times that of prior art specula has been obtained.

The enhancements inter alia include:

A. a single hinge pin lead slot 22 as discussed in conjunction with FIG. 11 above and formed by the side core method;

B. the use of stronger plastics such as polycarbonate;

C. positioning the threaded bolt or screw 320 higher up on the handle members 326 and 324 of speculum 310, as discussed and shown in conjunction with FIG. 13 above;

D. buttressing the speculum by thickening the plastic in selected regions of speculum 10 denoted as A through F in FIGS. 4 through 7 and FIG. 9;

E. a pair of struts 248 positioned over the distal curvature between the dorsal blade member 212 and the dorsal handle member 226 as in dorsal member 2112 of FIG. 14;

F. a threaded bolt or screw with rectangular 550B or trapezoidal 550A threads shown in FIGS. 16B and 16A, respectively;

G. a ridge 450 of plastic above hinge holes 430, as discussed in conjunction with FIGS. 15A and 15C above;

H. adding an extension 452 extending proximally from the proximal surface of the upper region of ventral handle member 424, as discussed in conjunction with FIGS. 15A and 15B above.

Plastic specula constructed according to the present invention have a destructive testing strength of at least about 6 kg, and therefore like metal specula they can be used in long duration gynecological procedures, including surgical procedures. Additionally, it is useful for gynecological out-patient procedures for which weaker specula are unsuited. These include coloscopy and IUD insertion.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. Therefore, it will be appreciated by persons skilled in the art that the present invention is not limited by what has been particularly shown and described herein above. Rather the scope of the invention is defined by the claims that follow.

What is claimed is:

1. A vaginal speculum comprising:
a molded plastic dorsal member having a rounded dorsal blade member formed integrally with a dorsal handle member, and having a first hinge location formed therealong on each lateral face of said dorsal member;
a molded plastic ventral member having a rounded ventral blade member formed integrally with a ventral handle member, and having a second hinge location formed therealong on each lateral face of said ventral member, said second hinge locations for interconnection with said first hinge locations thereby to form a hinge for facilitating relative angular translation between said dorsal member and said ventral member, about an axis extending transversely through said hinge, adapted to support thereat shear force components normal to said axis; and
a locking mechanism in mechanical communication with said dorsal and ventral handle members operable to lock the dorsal blade member and the ventral blade member in a user-selected position with respect to each other, and
wherein said hinge is reinforced so as to facilitate resistance to shear force components of a magnitude more normally associated with vaginal specula formed of metal.

2. A vaginal speculum according to claim 1 wherein said locking mechanism further comprises an at least partially threaded screw that is joined to and extends from said dorsal handle member through said ventral handle member and having a nut positioned thereon for holding said ventral and dorsal blade members in a predetermined user-selected position with respect to each other.

3. A vaginal speculum according to claim 2 wherein said screw has threads which form a profile chosen from a group of profiles consisting of a rectangular profile and a trapezoidal profile.

4. A vaginal speculum according to claim 3 wherein said screw and nut have at least one threaded region for facilitating ease of locking and unlocking said blade members in their predetermined user selected positions.

5. A vaginal speculum according to claim 2 wherein said screw has a head for preventing accidental dislodgement of said nut.

6. A vaginal speculum according to claim 2 wherein said screw is positioned near the points of maximum bending of said blade members.

7. A vaginal speculum according to claim 1 wherein said plastic is a high stress resistant plastic suitable for supporting stresses in excess of at least a force of 6 kgs without bending and without fracturing.

8. A vaginal speculum according to claim 7 wherein said high stress resistant plastic is polycarbonate.

9. A vaginal speculum according to claim 7 wherein said high stress resistant plastic is a transparent plastic.

10. A vaginal speculum according to claim 1 wherein each of said first hinge locations includes a hinge pin and each of said second hinge locations includes a hinge hole.

11. A vaginal speculum according to claim 10 wherein said hinge pins are mushroom-head pins.

12. A vaginal speculum according to claim 10 wherein on both of said lateral faces of said dorsal member proximal to said ventral member there is a thickening of the plastic in the neighborhood around said hinge holes.

13. A vaginal speculum according to claim 12 wherein each of said faces also has a hinge pin lead slot leading to said hinge holes from an edge of said dorsal member.

14. A vaginal speculum according to claim 13 wherein said hinge pin lead slot is formed by side core injection molding.

15. A vaginal speculum according to claim 10 wherein said ventral member includes at least one of the following structural features:
thickening of said plastic at a distal curved surface substantially at the point where said ventral blade member and said ventral handle member merge; and
thickening of said plastic at least one proximal surface of said ventral member substantially at the point where said ventral handle member and said ventral blade member merge and positioned below the level of said hinge pins.

16. A vaginal speculum according to claim 15 wherein said dorsal member includes at least one of the following structural features:
thickening of said plastic at a distal curved surface substantially at the point where said dorsal blade member and said dorsal handle member merge and positioned above the level of said hinge holes;
thickening of said plastic at least one curved surface positioned below the level of said hinge holes on said distal surface of said dorsal member; and
struts positioned along said distal curved surface of said dorsal member substantially in the region where said dorsal blade member and said dorsal handle member merge.

17. A vaginal speculum according to claim 10 wherein said ventral handle member further includes an extension so that when said ventral and dorsal handle members are brought together separating said ventral and dorsal blade members said extension is supported by said dorsal handle member thereby assisting in spreading the forces over the entire speculum, and wherein said dorsal member further includes a support ridge above said hinge holes therein, so that when said speculum is under load said speculum does not fracture in the region of said holes.

18. A vaginal speculum according to claim 1 wherein each of said second hinge locations includes a hinge pin and each of said first hinge locations includes a hinge hole.
